(19) United States
(12) Patent Application Publication
(10) Pub. No.: US 2022/0409498 A1
(43) Pub. Date: Dec. 29, 2022

(54) COMPOSITION FOR SUNSCREEN COSMETICS

(71) Applicant: FE:I BEAUTY TECH, INC., New York, NY (US)

(72) Inventor: Xi Huang, Montvale, NJ (US)

(21) Appl. No.: 17/902,741

(22) Filed: Sep. 2, 2022

Related U.S. Application Data
(63) Continuation-in-part of application No. PCT/US2021/033745, filed on May 21, 2021.
(60) Provisional application No. 63/029,039, filed on May 22, 2020.

(51) Int. Cl. A61K 8/19; A61Q 17/04; A61K 8/27; A61K 8/29; A61K 8/98

(52) U.S. Cl. CPC A61K 8/19; A61Q 17/04; A61K 8/27; A61K 8/29; A61K 8/987; A61K 2800/51

(57) ABSTRACT

The present invention relates to a sunscreen cosmetic for application to a user's skin. The sunscreen cosmetic is provided with a mineral including zinc oxide, titanium dioxide, or a combination of both. The sunscreen cosmetic also includes a high buffering-capacity agent configured to react with an external acidic source coming in contact with the cosmetic and to inhibit release of zinc ions from the zinc oxide and/or to inhibit release of titanium ions from the titanium dioxide.

### CROSS-REFERENCE TO RELATED APPLICATION

This is a continuation-in-part of International Patent Application No. PCT/US2021/033745 filed May 21, 2021, entitled COMPOSITIONS FOR SUNSCREEN COSMETICS, which claims priority to and the benefit of U.S. Provisional Patent Application No. 63/029,039, filed May 22, 2020, entitled COMPOSITIONS FOR SUNSCREEN COSMETICS, the disclosures of which are incorporated herein by reference in their entirety.

### FIELD OF THE INVENTION

The present invention relates to the fields of sunscreen cosmetics, methods for making and using sunscreen cosmetics, and methods for preventing toxic effects of sunscreen cosmetics containing minerals.

### BACKGROUND OF THE INVENTION

Many different sunscreen cosmetic compositions have been developed in the past. The sunscreen cosmetic compositions described herein inhibit toxic effects of sunscreen cosmetics containing minerals.

### SUMMARY OF THE INVENTION

In one embodiment, a high buffering-capacity (“HBC”) agent is incorporated into a sunscreen cosmetic having an oxide-based mineral. In one embodiment, the sunscreen cosmetic includes a transition metal oxide-based mineral. In one embodiment, the sunscreen cosmetic includes zinc oxides (ZnO) and/or titanium dioxides (TiO₂). In one embodiment, the sunscreen cosmetic includes a composition in a hydrated form (e.g., composition contains at least enough liquid phase to provide a medium or mechanism for transferring ions between zinc/titanium oxides and HBC agent such as an aqueous phase or a water- and/or oil-based emulsion). In one embodiment, the sunscreen cosmetic includes a composition in a hydrated non-anhydrous form (e.g., zinc sulfate heptahydrate (ZnSO_{4.}7H₂O)). In one embodiment, the sunscreen cosmetic includes a composition in a hydrated anhydrous form (e.g., ethylene glycol (OHCH₂CH₂OH) or other glycols suitable for use in skincare product formulations). In one embodiment, the sunscreen cosmetic includes a composition for a cream, a lotion, a spray, a gel, a foam, a stick, or other topical composition that can be absorbed into skin. In one embodiment, the HBC agent includes at least one of pearl powder and an analog thereof (e.g., one with high alkaline cation from the second column of the periodic table (e.g., alkaline earth metals) but weak acid (e.g., pKa range from 3 to 6)). In another embodiment, the HBC agent includes at least one of pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof.

In one embodiment, a sunscreen cosmetic composition includes oxide-based minerals and a high buffering-capacity (“HBC”) agent. In one embodiment, the sunscreen cosmetic composition includes transition oxide-based minerals and the HBC agent. In one embodiment, the sunscreen cosmetic composition includes zinc oxides and/or titanium dioxides and the HBC agent. In one embodiment, the HBC agent includes at least one of pearl powder and an analog thereof (e.g., one with high alkaline cation from the second column of the periodic table (e.g., alkaline earth metals) but weak acid (e.g., pKa range from 3 to 6)). In another embodiment, the high buffering capacity agent includes at least one of pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof.

The inventor herein has discovered, surprisingly and unexpectedly, that under an acidic condition, zinc oxides and titanium dioxide in cosmetics can be solubilized and release “free” zinc ions and titanium ions, which can be absorbed in the skin and can have toxic effects (e.g., oxidative or carcinogenic). In one embodiment, when an external acidic material is introduced to the sunscreen cosmetic applied to skin, the HBC agent is configured to maintain the pH of the sunscreen cosmetic at or near neutral (e.g., pH 7), thereby inhibiting solubilization of the zinc oxides and titanium dioxides contained in the sunscreen cosmetic and inhibiting release of “free” zinc and titanium ions therefrom. In one embodiment, the external acidic material can be an endogenous source, such as the skin and/or sweat excreted therefrom, and/or an exogenous source, such acidic rain. The HBC agent therefore keeps the sunscreen cosmetic safe for use by a user.

### BRIEF DESCRIPTION OF THE DRAWINGS

For a better understanding of the present invention, reference is made to the following detailed description of various exemplary embodiments considered in conjunction with the accompanying drawings, in which:

FIG. 1 shows the results of an example illustrating the release of water soluble zinc ions and titanium ions in commercially available mineral sunscreens;

FIG. 2 shows the results of an example illustrating the effect of acidic conditions on the release of zinc ions and titanium ions in commercially available mineral sunscreens;

FIG. 3 shows the results of an example illustrating the effects of varying amounts of calcite on the solubilization of zinc oxide in acidic conditions;

FIGS. 4A and 4B show the results of an example illustrating the effects of varying amounts of calcite on the inhibition of zinc oxide solubilization by consuming acid first and then release calcium ions in low concentration acidic conditions; and

FIG. 5 shows the results of an example illustrating the effects of varying amounts of calcite on the buffering capacity of mineral sunscreens.

FIG. 6 shows the results of an example illustrating the effects of ion exchange between Zn²⁺ and Ca²⁺ on the surface of the calcium carbonate.

### DETAILED DESCRIPTION OF EXEMPLARY EMBODIMENTS

Embodiments are now discussed in more detail referring to the drawings that accompany the present application. It is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

In one embodiment, a high buffering-capacity (“HBC”) agent is incorporated into a sunscreen cosmetic or a sunscreen composition containing zinc oxides (ZnO) or titanium dioxides (TiO₂). In one embodiment, the sunscreen cosmetic includes a composition in a hydrated form (e.g., composition contains at least enough liquid phase to provide a medium or mechanism for transferring ions between zinc/titanium oxides and HBC agent such as an aqueous phase or a water- and/or oil-based emulsion). In one embodiment, the sunscreen cosmetic includes a composition in a hydrated non-anhydrous form (e.g., zinc sulfate heptahydrate (ZnSO₄.7H₂O)). In one embodiment, the sunscreen cosmetic includes a composition in a hydrated anhydrous form (e.g., ethylene glycol (OHCH₂CH₂OH) or other glycols suitable for use in skincare product formulations). In a further embodiment, the sunscreen cosmetic includes a composition for a cream, a lotion, a spray, a gel, a foam, a stick, or other topical composition that can be absorbed into skin. In another embodiment, the present invention is useful in any cosmetic or sunscreen containing zinc oxides or titanium dioxides.

As used herein, the terms “high buffering-capacity agent”, “HBC agent”, and “buffering agent” means an agent, compound or material configured to react preferentially with an external acidic source or material and to inhibit (i.e., reduce zinc or titanium ion release such that the amount of zinc or titanium ion released from compositions with the buffering agent is less than the amount of zinc or titanium ion released from compositions that do not include the buffering agent) release of zinc ions from zinc oxides or release of titanium ions from titanium dioxides in their associated sunscreen cosmetics. In one embodiment, a surface of particles of the HBC agent is hydrophilic. In one embodiment, the HBC agent is a non-diffusible (e.g., water insoluble) agent, compound, or material. In one embodiment, the HBC agent may reduce release of zinc or titanium ions by about 0.2% or more. In another embodiment, the HBC agent may reduce release of zinc or titanium ions by about 0.2% or less. In one embodiment, the HBC agent may reduce release of zinc or titanium ions by about 28% or more. In another embodiment, the HBC agent may reduce release of zinc or titanium ions by about 94% or less. This also includes absorptions of zinc ions and titanium ions already released in the mineral zinc oxide and titanium dioxide sunscreens to form water insoluble zinc and titanium complexes (e.g., ZnₓCa(CO₃)ₓ₊₁ or TiₓCa(CO₃)₂ₓ₊₁) on the surface of the HBC agents and, thus, reduce the bioavailability of zinc and titanium ions. The external acidic source or material is a biological and/or environment source or material that is not part of the cosmetic itself, but mixes or otherwise comes in contact with the sunscreen cosmetic when it is applied to a user's skin. Examples of the external acidic material includes the user's skin, sweat, acid rain, an acidic beauty product, etc.

In sunscreen cosmetics, minerals, for example, zinc oxides or titanium dioxides, can be used as inorganic physical sun blockers. Furthermore, mineral based sunscreen cosmetics that include zinc oxides or titanium dioxides may be preferred over organic sunscreen cosmetics because mineral based sunscreen cosmetics may have limited skin penetration, less skin irritation and sensitization, and broad-spectrum UVB protection. Additionally, minerals such as zinc oxides and titanium dioxides have been considered relatively inert.

The inventor herein has discovered, surprisingly and unexpectedly, that when subjected to an acidic condition, zinc oxides and titanium dioxides in sunscreen cosmetics can be solubilized and release “free” zinc and titanium ions. As a result, conventional sunscreen creams, sunscreen lotions, and other sunscreen cosmetics containing zinc oxides or titanium dioxides could have toxic effects. The inventor herein has, surprisingly and unexpectedly, discovered that adding a buffer with a high buffering capacity can inhibit zinc and titanium solubilization and oxidant formation in sunscreen cosmetics.

In one embodiment, when the sunscreen cosmetic is exposed to an external acidic material or source (e.g., when it is applied to a person's skin, or when it comes in contact with sweat or acidic rain), the HBC agent is configured to react with the external acidic material or source and to inhibit release of zinc ions from zinc oxides and release of titanium ions from titanium dioxides in the sunscreen cosmetic. In one embodiment, the HBC agent is configured so as to interact with the external acid material or source and to neutralize same. In one embodiment, the HBC agent is configured to inhibit the external acidic material or source from significantly affecting (e.g., lowering more than 0.1 pH) the original pH of the sunscreen cosmetic (i.e., the pH of the sunscreen cosmetic prior to its exposure to the external acidic material or source or prior to its application to a person's skin). In one embodiment, the HBC agent is configured to inhibit the external acidic material or source and allows the sunscreen cosmetic to have a pH that remains substantially stable (e.g., the pH of the sunscreen cosmetic prior to its exposure to the external acid material or source or prior to its application to a person's skin remains within one pH upon exposure to an external acidic or source). In one embodiment, the HBC agent is configured to react with the external acidic material or source and to maintain the original pH of the sunscreen cosmetic substantially constant (e.g., within one pH of the original pH). In one embodiment, the HBC agent is configured to react with the external acidic source or material and to maintain the pH of the sunscreen cosmetic substantially at neutral (e.g., pH range of about 6.5 to about 7.5). In one embodiment, the HBC agent is configured to react with the external acidic material or source and to maintain the pH of the sunscreen cosmetic substantially at neutral (e.g., pH range of about 6.5 to about 7.5) or near neutral (e.g., within two pH of pH 7). In one embodiment, the HBC agent is configured to react with the external acidic material or source and to maintain the pH of the sunscreen cosmetic between about 6.8 and about 7.2. In another embodiment, the HBC agent is configured to react with the external acidic material or source and to maintain the pH of the sunscreen cosmetic between about 6.5 and about 8. In other embodiments, the HBC agent is configured to react with the external acidic material or source and to maintain the pH of the sunscreen cosmetic between about 6 and about 10 or above 6.0.

In one embodiment, the HBC agent includes pearl powder and/or calcite powder. Pearl and calcite contain high levels of calcium carbonate (CaCO₃). In one embodiment, pearl powder may contain at least 90% calcium carbonate with the remaining percentage of the pearl powder including proteins, amino acids, and peptide. In another embodiment, the HBC agent may be any one of analogs of pearl or calcite powder (e.g., one with high alkaline cation from the second column of the periodic table (e.g., alkaline earth metals) but weak acid (e.g., pKa range from 3 to 6)). Non-limiting examples of such analogs include pearl powder, calcium carbonate, calcium citrate, calcium phosphate, calcium silicate, calcium molybdate, calcium tungstate, magnesium carbonate, magnesium phosphate, magnesium silicate, magnesium selenate, barium carbonate, barium phosphate, barium silicate, barium oxalate, barium molybdate, barium manganate, barium selenate, beryllium carbonate, beryllium phosphate, beryllium silicate, strontium carbonate, strontium phosphate, strontium silicate, strontium molybdate, strontium tungstate, strontium selenate, and a combination thereof. Pearl powder with a combination of calcium carbonate and amino acids, peptides, and proteins may be softer on skin than calcite and other HBC agents. Calcium-based HBC agents are more fine-tuned than pearl powder and calcite, and are particularly suitable because skin contains high levels of calcium ions. Additionally, dissolution of calcium carbonate will release calcium ion and carbon dioxide (CO₂), which has limited effects on the skin.

In one embodiment, the amount of the HBC agent included in the sunscreen cosmetic ranges from about 0.01% (w/w) to about 10% (w/w) relative to the overall weight of the cosmetic. In another embodiment, the HBC agent amount ranges from about 2.0% (w/w) to about 10% (w/w). In yet another embodiment, the HBC agent amount ranges from about 0.5% (w/w) to about 5% (w/w). In a further embodiment, the HBC agent amount ranges from about 1% (w/w) to about 2% (w/w).

In one embodiment, the sunscreen cosmetic can include conventional components that are included in a sunscreen cosmetic product. Such components are readily understood by a person of ordinary skill in the art. Examples of such components are disclosed in U.S. Patent Application Publication Nos. 2010/0183528 A1 published Jul. 22, 2010; and 2012/0269753 A1 published Oct. 25, 2012, the disclosures of all of which are incorporated herein by reference in their entireties.

In one embodiment, the HBC agent is in powder form. In one embodiment, the sizes of the particles of the HBC agent range from about 0.05 μm to about 30 μm. In one embodiment, the sizes of the particles of the HBC agent range from about 0.1 μm to about 30 μm. In one embodiment, the sizes of the particles of the HBC agent range from about 0.3 μm to about 20 μm. In one embodiment, the sizes of the particles of the HBC agent range from about 0.3 μm to about 15 μm. In one embodiment, the sizes of the particles of the HBC agent range from about 0.3 μm to about 10 μm. In one embodiment, the sizes of the particles of the HBC agent range from 0.3 μm to about 5 μm. In one embodiment, the sizes of the particles of the HBC agent range from about 0.3 μm to about 2 μm. In one embodiment, the sizes of the particles of the HBC agent range from about 0.03 μm to about 1 μm. In other embodiments, the sizes of the particles of the HBC agent are greater than about 0.05 μm, about 0.1 μm, or about 0.3 μm and/or less than about 30 μm, about 20 μm, about 15 μm, about 10 μm, or about 5 μm.

In one embodiment, the HBC agent can be prepared in any method known in the art for preparing powder. For instance, pearls can be crushed, grinded or milled into fine powder using a conventional blender, grinder, mill, etc.

In one embodiment, the HBC agent is not treated with any agent. For example, the HBC agent is not surface-treated with any agent, such as hydrophobilizing agent, such that it is hydrophilic and can readily react with protons (H+). In one embodiment, the HBC agent is prepared as a metal ion absorbent by performing ion exchange. In one embodiment, the HBC agent is formed as fine particles having a large surface area for absorbing zinc and titanium ions.

In one embodiment, the HBC agent is added to a sunscreen cosmetic composition including zinc oxides and/or titanium dioxides to form a desired sunscreen cosmetic. In one embodiment, the sunscreen cosmetic includes a composition in a hydrated form (e.g., composition contains at least enough liquid phase to provide a medium or mechanism for transferring ions between zinc/titanium oxides and HBC agent such as an aqueous phase or a water- and/or oil-based emulsion). In one embodiment, the sunscreen cosmetic includes a composition in a hydrated non-anhydrous form (e.g., zinc sulfate heptahydrate (ZnSO₄.7H₂O)). In one embodiment, the cosmetic includes a composition in a hydrated anhydrous form (e.g., ethylene glycol (OHCH₂CH₂OH) or other glycols suitable for use in skincare product formulations). In one embodiment, the cosmetic includes a composition for a cream, a lotion, a spray, a gel, a foam, a stick, or other topical composition that can be absorbed into skin. In one embodiment, the sunscreen cosmetic composition can be made using any methods known in the art. For instance, a sunscreen cosmetic preparation (e.g., an emulsion) containing zinc oxides or titanium dioxides can be prepared in a conventional manner, and then the HBC agent can be added to and homogenized with the sunscreen composition prior to performing finishing steps, such an optional drying step, etc. In one embodiment, the HBC agent can be prepared in a hydrated phase (an aqueous phase or as an emulsion). For instance, the HBC agent can be prepared in an aqueous phase by pre-dispersing the HBC agent in an organic solvent, and then adding to an oil phase, such as pentylene glycol, before mixing with a water phase. In one embodiment, the HBC agent can be prepared as an emulsion by pre-dispersing the HBC agent in a water phase and then adding to an oil phase. In one embodiment, the HBC agent in an aqueous phase or as an emulsion is added to a cosmetic composition containing including zinc oxides or titanium dioxides to form a desired sunscreen cosmetic, such as a cream, a lotion, a spray, a gel, a foam, a stick, or other topical composition that can be absorbed into skin.

In one embodiment, the sunscreen cosmetic composition is in a hydrated form (e.g., composition contains at least enough liquid phase to provide a medium or mechanism for transferring ions between zinc/titanium oxides and HBC agent such as an aqueous phase or a water- and/or oil-based emulsion). In one embodiment, the sunscreen cosmetic includes a composition in a hydrated non-anhydrous form (e.g., zinc sulfate heptahydrate (ZnSO₄.7H₂O)). In one embodiment, the sunscreen cosmetic includes a composition in a hydrated anhydrous form (e.g., ethylene glycol (OHCH₂CH₂OH) or other glycols suitable for use in skincare product formulations). In one embodiment, the hydrated form of the sunscreen cosmetic composition includes a fluid that provides a medium or mechanism for transferring ions between the zinc/titanium oxides and the HBC agent. The hydrated form improves the interaction between the zinc/titanium oxides and the HBC agent and the buffering effects of the HBC agent. Also, increasing the water content of the hydrated form of the sunscreen cosmetic composition can further improve the interaction between the zinc/titanium oxides and the HBC agent and the buffering effects of the HBC agent. In addition, the HBC agent in a hydrated phase improves the uniformity of the HBC agent in the sunscreen cosmetic composition (e.g., homogenizes). The homogenized composition has a pH that remains substantially stable (e.g., the pH of the sunscreen cosmetic composition remains within one pH) when the sunscreen cosmetic composition is in a container (e.g., a bottle containing the sunscreen cosmetic composition). In one embodiment, the sunscreen cosmetic composition is prepared such that its pH is and/or remains substantially at about 7. In one embodiment, the sunscreen composition is prepared such that its pH is and/or remains at or above about 6.8. In one embodiment, the sunscreen cosmetic composition provides a stable formulation for containing the HBC agent.

The substantially stable, homogenized composition inhibits the formation of foam in the sunscreen cosmetic composition during storage in the container. For example, calcium carbonate can react with acids to produce CO₂ gas, creating foams in the formulation. In addition, the homogenous sunscreen cosmetic composition spreads the HBC agent uniformly across a user's skin when the sunscreen cosmetic composition is applied. Over time, the HBC agent remains uniformly spread across the user's skin, even if the liquid contents diminish due to evaporation. This improves the coverage of the HBC agent on the skin, which improves the protection against the release of zinc or titanium ions caused by the external acidic sources or materials. Also, the homogenous sunscreen cosmetic composition provides instant protection on the user's skin. The user's skin can produce acidic materials (e.g., protons (H+)) and the uniform application of the HBC agent is above to consume the acidic materials before the zinc/titanium oxides because the HBC agent has a stronger alkalinity.

In one embodiment, the HBC agent is configured such that when the sunscreen cosmetic composition is applied to a user's skin, the HBC agent is not absorbed into the skin, but remains on same to react with acidic sources or materials. In one embodiment, the HBC agent is water-insoluble and hence non-diffusible such that it remains on (i.e., is not diffused into) the skin to provide long-lasting protection against acidic sources or materials.

Provided below are a more detailed discussion of the discoveries made by the inventor herein and various examples associated with same.

Sunscreen cosmetics have been used to provide protection against the effects of solar ultraviolet (UV) radiation, including both UVB and UVA radiation, on a user's skin. For example, UVA and UVB radiation can produce reactive oxygen species (ROS) that activate different matrix metalloproteinases, which damage collagen and other matrix proteins. This damage can result in loss of skin elasticity. The damage caused by UVA and UVB, collectively, is called photo-aging (i.e., formation of wrinkles on skin). In the last decennia, only sunscreen cosmetics containing both UVB and UVA filters have been produced. Minerals like zinc oxide and titanium dioxide can be used as inorganic physical sun blockers. They may be preferred above organic sunscreens because of limited skin penetration, absence of skin irritation and sensitization, and a broad-spectrum UV protection. Additionally, zinc oxides and titanium dioxides have been considered to be relatively inert.

Despite the foregoing conventional belief, the inventor herein has discovered, surprisingly and unexpected, that mineral based sunscreen cosmetics undergo chemical interactions with intrinsic and extrinsic environments. For example, the acidic pH in skin and in the sweat under the heat, and acid rain due to environmental pollution can solubilize the zinc oxides in the sunscreen cosmetics and release zinc ions for skin absorption as follows:

ZnO+2H⁺→Zn²⁺+H₂O (1)

To a lesser degree, the acidic condition could lead titanium dioxide in the sunscreen cosmetics to release titanium ions for skin absorption as follows:

TiO₂+4H⁺→Ti⁴⁺+2H₂O (2)

In addition, the inventor herein has discovered, surprisingly and unexpected, that zinc ions and titanium ions already released in mineral based sunscreen cosmetics can be absorbed by the HBC agents to form water insoluble zinc and titanium complexes (e.g., ZnₓCa(CO₃)ₓ₊₁ or TiₓCa(CO₃)₂ₓ₊₁). This absorption further reduces the bioavailability of zinc and titanium ions in the mineral based sunscreen cosmetics. The chemical reaction for the absorption of zinc ions can be written as follows:

Zn²⁺+2CaCO₃→ZnCa(CO₃)₂+Ca′

The chemical reaction for the absorption of titanium ions can be written as follows:

Ti⁴⁺+3CaCO₃→TiCa(CO₃)₃+2Ca²⁺

Since the natural opaqueness of these micro-sized mineral sunscreens can be eliminated without reducing their UV blocking efficacy by utilizing nano-sized zinc oxide and titanium dioxide particles, the sunscreen industry tend to use nanoparticles of the mineral sunscreens. Because of the smaller sizes, the larger surface areas allow more access to the acidic environment, which could make nanoparticles of zinc oxide and titanium dioxide even more susceptible to acid solubilization. Thus, the dissolved zinc and titanium ions may play an important role in the toxic effects of zinc oxide and titanium dioxide particles. For example, based on the experimental evidence from animal inhalation studies, titanium dioxide nanoparticles are classified as “possible carcinogenic to humans” by the International Agency for Research on Cancer and as occupational carcinogen by the National Institute for Occupational Safety and Health. Therefore, titanium dioxide is prohibited to use in a sprayed sunscreen in the European Union.

In addition, dissolution of zinc oxide and titanium dioxide can make these metal ions more bioavailable for cell uptake and, thus, cell toxicity. Another example is that zinc ions can help iron-catalyzed oxidation, known as Fenton reaction, to be more complete and more damaging.

### Example 1

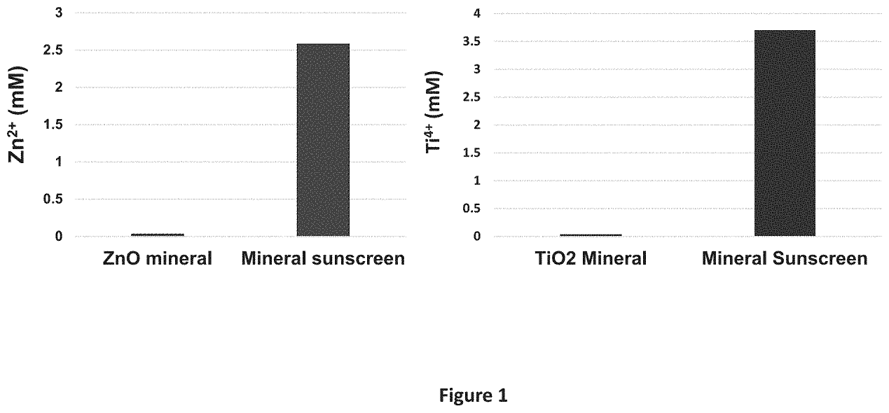

The water solubility, and thus, bioavailability of zinc ions (Zn²⁺) and titanium ions (Ti⁴⁺) in commercial mineral sunscreen was examined. FIG. 1 shows results from suspending a hydrating sunscreen from CeraVe in water at 75 mg/ml. The CeraVe sunscreen has a spectrum SPF 50 and contains 9% titanium dioxide (TiO₂) and 7% zinc oxide (ZnO). After suspension of the CerVe sunscreen, it released 2.7 mM Zn2+ ions and 3.7 mM Ti4+ ions. Assuming if all ZnO and TiO₂ were solubilized, these results indicate that the concentrations are equal to 0.28% ZnO and 0.39% TiO₂ particles solubilized. This gives a total concentration of Zn2+ and Ti4+ at 921 mM and 931 mM, respectively. It is interesting to note that mineral ZnO alone (IWAS, Japan) did not release Zn2+ in water (FIG. 1, 1st bar). These results suggest that mineral sunscreens, such as CeraVe, contain water soluble Zn2+ and Ti4+ ions that are readily bioavailable for toxic effects on the skin or after inhalation.

### Example 2

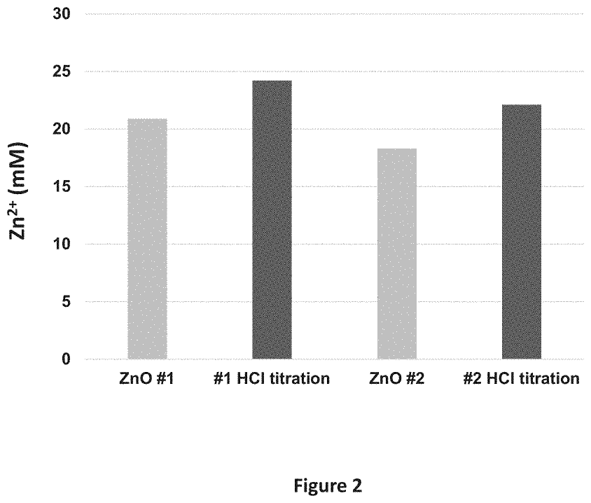

As shown in FIG. 1, pure ZnO mineral as a raw material did not release Zn2+ in water. This was probably due to surface treatment. To further investigate this result, two pure ZnO minerals (#1: IWAS from Japan and #2: K. S. Pearl from South Korea) were suspended in 40 ml of 50% tetraethyl glycol (TEG) in water. FIG. 2 shows that both pure ZnO sunscreens #1 and #2 released large amounts of Zn2+ ions, 20.9 mM and 18.3 mM respectively. To demonstrate whether there is sufficient protection against the acidic pH of the skin, the sweat, and acid rain, 800 μl 100 mM HCl were used to titrate the suspensions. The pHs of the suspensions were slightly changed from 6.46 to 6.39 for sample #1 and 6.63 to 6.59 for sample #2, but the Zn2+ ion concentrations were increased from 20.9 mM to 24.2 mM or 15.8% for sample #1 and from 18.3 mM to 22.1 mM or 20.8% for sample #2, respectively. These results indicate that, despite surface treatment, which makes the mineral sunscreens such as ZnO and TiO₂ water insoluble, ZnO and TiO₂ can still interact with the acidic pH in a mixture of water and organic solvent, such as 50% TEG. These results are significant because any emulsion with a mixture of water phase and oil phase will render the mineral sunscreen accessible to acidic conditions This may result in solubilization of the mineral particles, which could lead to unsafe use of the mineral sunscreens.

### Example 3

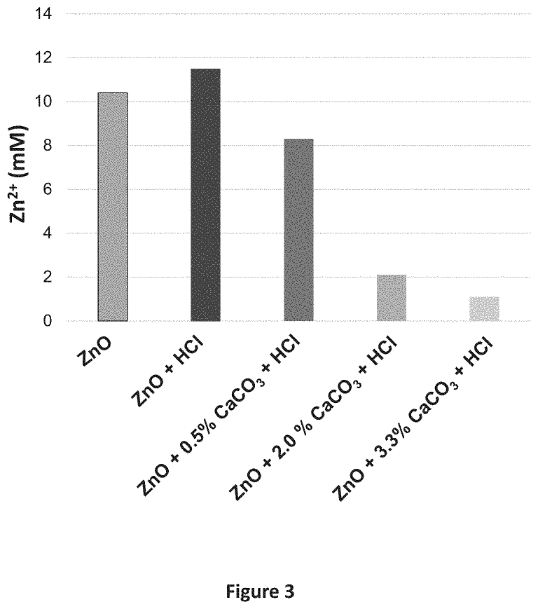

The effects of adding calcite into mineral sunscreens, in particular calcite's effects on the buffering capacity of the mineral sunscreens, were examined. This Example investigates the effects of calcite on inhibiting Zn2+ release from ZnO in the presence of acid. FIG. 3 shows that pure ZnO released large amounts of Zn2+ and titration of the suspension with 800 μl of 100 mM HCl further increased the amount of Zn2+ released by 10.6%. Interestingly, the addition of calcium carbonate prevented the acid-induced Zn2+ release and this prevention is calcium carbonate dose-dependent.

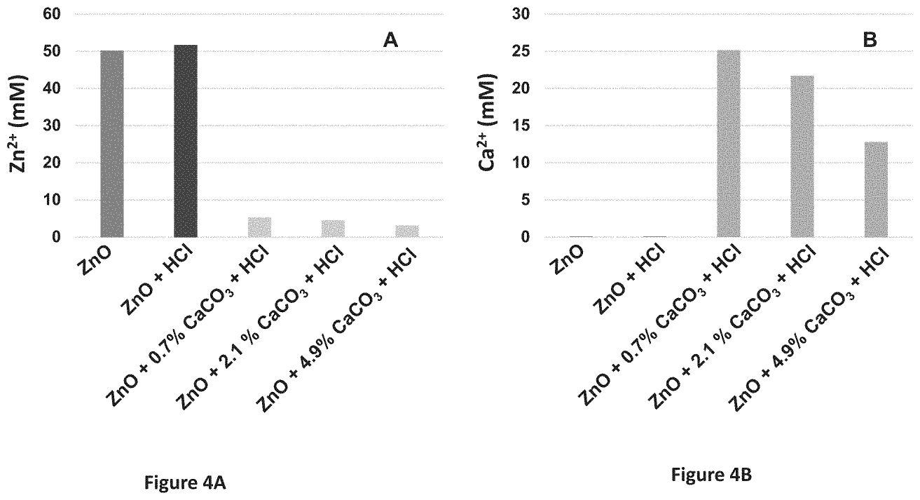

To further demonstrate that inhibited Zn2+ release by HCl is due to the preferred acid consumption by CaCO₃ over ZnO, the following formulation were examined: 0.7%, 2.1%, and 4.9% of CaCO₃ (W/W) mixed with a ZnO level equal to the ZnO level in the CeraVe mineral sunscreens at 7% (W/W). This resulted in weight ratios of CaCO₃ to ZnO at 10:1, 10:3, and 10:7. Additionally, a lower concentration of HCl at 50 mM for titration was used. Beside Zn2+ measurements, Ca2+ levels in the suspensions were also measured. FIG. 4A shows that 50 mM HCl was able to increase the amount of released Zn2+ by only 3%. By comparing Zn2+ concentrations of the same pure ZnO mineral sunscreens without any treatments among FIGS. 2, 3, and 4, there were big variations of the background levels of Zn2+ ions, suggesting heterogeneity of the sample. However, the trend is the same. Interestingly, calcium carbonate inhibited the acid induced Zn2+ release. FIG. 4B shows that lower concentration of HCl had a greater ability to release more Ca2+ in 0.7% CaCO₃ than in 2.1% and 4.9% CaCO₃, respectively, despite the fact that the absolute amount of HCl used in FIG. 3 is the same as in FIG. 4. These results indicate that higher levels of CaCO₃ may be necessary for prolonged exposure to weak acid conditions. Moreover, because the amount of acid was limited (FIG. 4A), it would be expected that amount of Ca2+ released in FIG. 4B should be the same if calcite completely reacts with the limited acid and the amount of Zn2+ in FIG. 4A should be the same as well. In contrast and unexpectedly, the amount of Ca2+ released was lower in higher concentration of calcite but the bioavailable Zn2+ was lower in higher concentrations of calcite. These results strongly indicate that, when the amount of acid is limited, the addition of calcite into the mineral sunscreen can absorb Zn2+ ions and Ti4+ ions on the surface of the calcite to form water insoluble zinc and calcium carbonate complexes (e.g., ZnₓCa(CO₃)ₓ₊₁), further reducing the amount of water soluble Zn2+ and Ti4+ in the sunscreens.

### Example 4

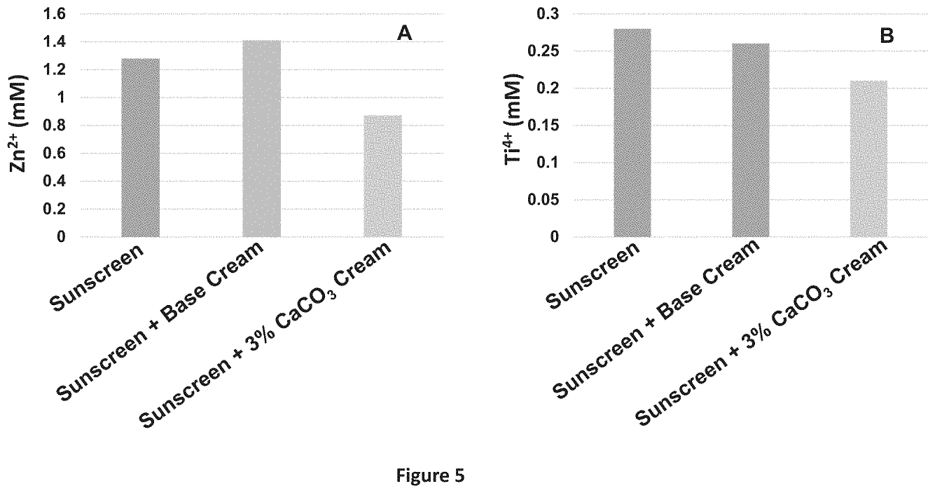

CaCO₃ has been shown that it can inhibit Zn2+ release from ZnO. It is unclear whether this inhibition can occur in commercially available sunscreens. Thus, base cream and the same cream containing 3% CaCO₃ was prepared so that these will be compatible when mixing with the mineral sunscreen. By mixing the base cream and 3% CaCO₃-containing base cream with the commercially available mineral sunscreen (CeraVe, SPF 50) at a weight ratio of 50:50 (w/w), the mixtures were suspended in 50% ethanol at 50 mg/ml overnight. FIG. 5 shows that the mineral sunscreen released Zn2+ and Ti2+ in ethanol as previously observed in TEG. Base cream had no significant effect on Zn2+ and Ti4+ release. Interestingly, 3% CaCO₃ significantly reduced levels of Zn2+ and Ti4+. These results indicate that premixing of CaCO₃ with mineral sunscreens will have the ability to inhibit Zn2+ and Ti4+ release and thus, reduce their bioavailability and toxicity.

### Example 5

Because of the high buffering capacity and the alkaline nature of calcium carbonate, the first mechanism of Zn and Ti ion removal is to precipitate them by the following reactions:

Zn²⁺+2 OH⁻→Zn(OH)₂ 1) and

Ti⁴⁺+4 OH⁻→Ti(OH)₄ 2)

Another mechanism, which is unexpected, is ion exchange between Zn²⁺ and Ca²⁺ on the surface of the calcium carbonate. To illustrate these ion exchange reactions, various concentrations of ZnCl₂ were incubated with different concentration of calcium carbonate for 30 minutes, 1 hour, and overnight. After incubations, a calcium-sensitive dye, Arsenazo III (Sigma-Aldrich, #A92775) was used to measure the concentration of calcium ions released into the solution from the surface of calcium carbonate. Arsenazo III at 0.05% (5 mg/10 ml) was prepared in water. After adding 100 microliter (μl) per well, samples of ZnCl₂ and calcium carbonate mixture were centrifuged and 5 μl of the supernatant were taken from the incubation and added into each well. CaCl₂ was used as standard. After incubating for 5 mins at room temperature, Ca²⁺ was measured at 660 nm and calculated by comparing to the standard curve. (https://pubmed.ncbi.nlm.nih.gov/1597016/). To determine how much Zn²⁺ ions remain in the solution, dithizone (diphenylthiocarbazone, Sigma, #43820) is used for spectrophotometric measurement of Zn²⁺ ions. Dithizone was prepared at 0.05% solution in 95% ethanol (5 mg/10 ml). After adding 200 μl per well, 10 μl samples were added into each well. ZnCl₂ is used as standard. After incubating for 5 mins at RT, Zn2+ concentrations were measured at 490 nm and calculated after comparing it to the standard curve.

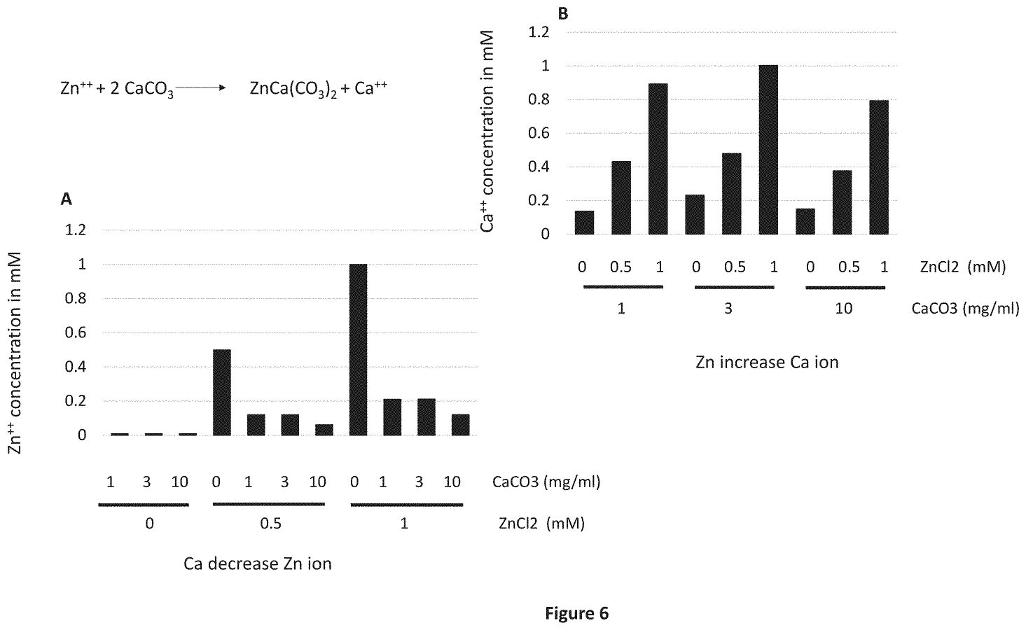

As shown in FIG. 6, after a 30 minute incubation time, CaCO₃ at 1%, 3%, and 10% can absorb 76% (0.12 mM remaining), 76% and 88% (0.06 mM Zn remaining) of Zn ions in a 0.5 mM ZnCl₂ solution. In a 1 mM ZnCl₂ solution, CaCO₃ at 1%, 3%, and 10% can absorb 79% (0.21 mM Zn remaining), 79%, and 88% (0.12 mM Zn remaining) of Zn ions. There are no significant differences among ion exchanges for 1 hour or overnight incubation (data not shown). Hydrophilic property of the calcium carbonate in our case facilitates the ion exchange mechanism. The foregoing results indicate that Ca²⁺ is released in Zn-dose dependent manner, suggesting an ion exchange mechanism.

It will be understood by those having ordinary skill in the art and possession of the present disclosure that the embodiments described herein are merely exemplary in nature and that a person skilled in the art may make many variations and modifications thereto without departing from the scope of the present invention. All such variations and modifications are intended to be included within the scope of the invention.

What is claimed is:

1. A method for inhibiting damage caused to a user's skin by a sunscreen cosmetic containing a mineral, said method comprising the step of:
applying a sunscreen cosmetic composition in a hydrated form to the user's skin, wherein the sunscreen cosmetic composition comprises a mineral including at least one of: zinc oxide, titanium dioxide, or a combination thereof, and a buffering agent, the buffering agent reacting with an external acidic source coming in contact with the sunscreen cosmetic composition and inhibiting release of zinc ions from the zinc oxide and/or inhibiting release of titanium ions from the titanium dioxide.

2. The method of claim 1, wherein the buffering agent reacts with the external acidic source such that the pH level of the sunscreen cosmetic composition remains substantially stable despite exposure to the external acidic source.

3. The method of claim 2, wherein the buffering agent reacts with the external acidic source and maintains the pH level of the sunscreen cosmetic composition substantially at neutral.

4. The method of claim 1, wherein the pH level of the sunscreen cosmetic composition is at or above about 6.8.

5. The method of claim 1, wherein the buffering agent includes at least one of pearl powder, calcium carbonate, calcium citrate, calcium phosphate, calcium silicate, calcium molybate, calcium tungstate, magnesium carbonate, magnesium phosphate, magnesium silicate, magnesium selenate, barium carbonate, barium phosphate, barium silicate, barium oxalate, barium molybate, barium manganate, barium selenate, beryllium carbonate, beryllium phosphate, beryllium silicate, strontium carbonate, strontium phosphate, strontium silicate, strontium molybate, strontium tungstate, strontium selenate, or a combination thereof.

6. The method of claim 1, wherein said buffering agent is prepared in a hydrated form.

7. The method of claim 1, wherein the buffering agent is included in the sunscreen cosmetic in an amount ranging from about 0.01% (w/w) to about 10% (w/w).

8. The method of claim 7, wherein the amount of the buffering agent ranges from about 0.01% (w/w) to about 2% (w/w).

9. The method of claim 1, wherein the buffering agent includes pearl powder having a size ranging from about 0.05 μm to about 30 μm.

10. The method of claim 1, wherein the sunscreen cosmetic includes one of a composition for a cream, a lotion, a spray, a gel, a foam, or a stick.

11. A method for inhibiting damage caused to a user's skin by a sunscreen cosmetic containing a mineral, said method comprising the step of:
preparing a sunscreen cosmetic composition having mineral, which includes zinc oxide, titanium dioxide, or a combination thereof, wherein said preparing step includes the step of including in said sunscreen cosmetic composition a buffering agent configured to react with an external acidic source coming in contact with the sunscreen cosmetic composition and inhibiting release of zinc ions from the zinc oxide and/or inhibiting release of titanium ions from the titanium dioxide.

12. The method of claim 11, wherein the
buffering agent reacts with the external acidic source such that the pH level of the sunscreen cosmetic composition remains substantially stable despite exposure to the external acidic source.

13. The method of claim 12, wherein the buffering agent reacts with the external acidic source and maintains the pH level of the sunscreen cosmetic composition substantially at neutral.

14. The method of claim 11, wherein the pH level of
the sunscreen cosmetic composition is at or above about 6.8.

15. The method of claim 11, wherein the buffering agent includes at least one of pearl powder, calcium carbonate, calcium citrate, calcium phosphate, calcium silicate, calcium molybate, calcium tungstate, magnesium carbonate, magnesium phosphate, magnesium silicate, magnesium selenate, barium carbonate, barium phosphate, barium silicate, barium oxalate, barium molybate, barium manganate, barium selenate, beryllium carbonate, beryllium phosphate, beryllium silicate, strontium carbonate, strontium phosphate, strontium silicate, strontium molybate, strontium tungstate, strontium selenate, or a combination thereof.

16. The method of claim 11, wherein said buffering agent is prepared in a hydrated form.

17. The method of claim 11, wherein the buffering agent is included in the sunscreen cosmetic in an amount ranging from about 0.01% (w/w) to about 10% (w/w).

18. The method of claim 17, wherein the amount of the buffering agent ranges from about 0.01% (w/w) to about 2% (w/w).

19. The method of claim 11, wherein the buffering agent includes pearl powder having a size ranging from about 0.05 μm to about 30 μm.

20. The method of claim 11, wherein the sunscreen cosmetic includes one of a composition for a cream, a lotion, a spray, a gel, a foam, or a stick.
